(19) United States
(12) Patent Application Publication
(10) Pub. No.: US 2013/0211543 A1
(43) Pub. Date: Aug. 15, 2013

(54) TISSUE SCAFFOLD WITH CONTROLLED DRUG RELEASE

(71) Applicants: Muwan Chen, Aarhus C (DK); Dang Quang Sven Le, Aarhus C (DK); San Hein, Reno, NV (US); Jørgen Kjems, Risskov (DK); Cody Eric Bünger, Auning (DK); Jens Vinge Nygaard, Hjortshoj (DK)

(72) Inventors: Muwan Chen, Aarhus C (DK); Dang Quang Sven Le, Aarhus C (DK); San Hein, Reno, NV (US); Jørgen Kjems, Risskov (DK); Cody Eric Bünger, Auning (DK); Jens Vinge Nygaard, Hjortshoj (DK)

(73) Assignees: REGION MIDTJYLLAND, Viborg (DK); AARHUS UNIVERSITET, Aarhus C (DK)

(21) Appl. No.: 13/880,204

(22) PCT Filed: Oct. 7, 2011
(86) PCT No.: PCT/DK2011/050381

§ 371 Date: Apr. 18, 2013

(30) Foreign Application Priority Data
Oct. 19, 2010 (DK) PA 2010 70443

(51) Int. Cl. A61F 2/02

(52) U.S. Cl. CPC A61F 2/02
USPC 623/23.72

(57) ABSTRACT

A three-dimensional hybrid scaffold capable of supporting cell activities such as growth and differentiation, and capable of controlled release of active pharmaceutical ingredients, characterized in that the scaffold comprises a first and a second biocompatible material, said first material shaped as a framework forming one or more open networks of voids, said second material comprising an ion exchange material, said ion exchange material being loaded with one or more active pharmaceutical ingredients.

### TECHNICAL FIELD OF THE INVENTION

The present invention relates to three-dimensional hybrid scaffolds with optimal mechanical properties and controlled drug-release profile for a therapeutic strategy based on tissue engineering.

### BACKGROUND OF THE INVENTION

Bone defects induced by primary tumor resection, trauma, or selective surgery have in many cases presented insurmountable challenges to the current gold standard treatment for bone repair. Recent advances in biology, medicine, and engineering have led to the discoveries of new therapeutic agents and novel materials for the repair of large bone defects caused by trauma, congenital defects, or bone tumors (Sohier, Daculsi et al.; Meijer, de Bruijn et al. 2007; Huang, Shi et al. 2010). These repair strategies often use degradable polymeric scaffolds for the controlled localized delivery of bioactive molecules to stimulate bone ingrowth as the scaffold degrades (Lee and Shin 2007; Wenk, Meinel et al. 2009).

Bioactive ceramic scaffolds can serve as a drug delivery vehicle due to their high affinity to dissolved drugs. However, the drug release profiles are difficult to control (Habraken, Wolke et al. 2007).

Biodegradable polymeric materials such as polycaprolactone (PCL) and poly (lactic-co-glycolic acid) (PLGA) can be used to control the local drug delivery; but they lack osteoconductivie and biological functions for cell attachment and tissue ingrowth (Zhu, Gao et al. 2002; Mei, Chen et al. 2005).

Doxorubicin, a well-known anticancer drug, is an anthracycline antibiotic with a broad spectrum antitumor activity to treat several types of cancer. However, treatment with doxorubicin is restricted due to several undesirable side effects like its cumulative-dose limit and cardiotoxicity (Kumar, Kirshenbaum et al. 2001; Takemura and Fujiwara 2007).

Hence, an improved tissue scaffold would be advantageous, and in particular a more efficient and/or reliable controlled localized delivery of bioactive molecules from the tissue scaffold would be advantageous.

### SUMMARY OF THE INVENTION

Thus, it is an object of the present invention to provide a scaffold with optimal mechanical properties and controlled drug-release profile for a therapeutic strategy based on bone tissue engineering.

Another object of the present invention is to develop a biocompatible implant that would be placed at the defect site during tumor resection surgery. This construct would provide adequate structural (mechanical) support, locally provide a controlled sustained release of a chemotherapeutic agent, and eventually promote tissue ingrowth.

In particular, it is an object of the present invention to provide a new type of active pharmaceutical ingredient (API) eluting tissue scaffold. The tissue scaffold is constructed by infiltrating a macro porous scaffold made by rapid prototyping with a micro porous clay matrix comprising the API of interest. The invention serves two purposes: to provide a long-term, localized API release, and to aid subsequent tissue regeneration. The 3D plotted scaffold gives not only the proper mechanical support but also the flexibility of producing different shapes and forms.

Thus, one aspect of the invention relates to a three-dimensional hybrid scaffold capable of supporting cell activities such as growth and differentiation, and capable of controlled release of active pharmaceutical ingredients, characterized in that the scaffold comprises a first and a second biocompatible material, said first material shaped as a framework forming one or more open networks of voids, said second material comprising an ion exchange material, said ion exchange material being loaded with one or more active pharmaceutical ingredients.

### BRIEF DESCRIPTION OF THE FIGURES

FIG. 1 shows a schematic representation of the composite scaffold preparation of groups A-D,

FIG. 2 shows the cumulative DOX release (%) at 37° C. in PBS (pH 7.4) for 2 months from: group B—pure DOX without clay scaffold, group C—DOX with clay scaffold and group D—modified clay/DOX carrier scaffold,

FIG. 3 shows element component analysis (EDX) of crystal-like extracellular matrix deposition observed on the surface of the group A scaffolds with 21-days culture,

FIG. 4 shows the quantification of dsDNA in cellular group A scaffolds by Picogreen assay on day 1, day 7, day 14 and day 21. The amount of DNA is expressed as mean±SD (n=4),

FIG. 5 shows the activity of alkaline phosphatase (ALP) enzyme on day 7, day 14 and day 21. The activity is expressed as mean±SD (n=4). Activity is indicated in nanomole p-nitrophenol/microgram DNA per minute (nmol/μg DNA*min),

FIG. 6 shows the calcium contents per scaffold on day 1, day 7, day 14, and day 21. The amount of calcium is expressed as mean±SD (n=4),

FIG. 7 shows von Kossa staining of scaffolds on day 1, day 7, day 14, and day 21,

FIG. 8 shows A: Cumulative DOX release (%) from premade composite scaffold loaded with DOX at 37° C. in PBS (pH 7.4) for 2 months; B: Cumulative BSA release from pure PCL scaffolds and composite scaffold at 37° C. in PBS (pH 7.4) for 1 month. Dashed lines represent the loaded amount of BSA in the scaffolds,

FIG. 9 shows cumulative Lysozyme release from pure PCL scaffolds and composite scaffolds at 37° C. in PBS (pH 7.4) for 1 month. Dashed lines represent the amount of Lysozyme loading in the scaffolds, and

FIG. 10 shows GFP silencing after 72 hours in three cancer cell lines grown beneath scaffolds loaded with three different siRNA transfection reagents targeted towards GFP. NTC: negative control, OM: oligofectamine with mismatch siRNA, OG: oligofectamine with targeted GFP siRNA, LM: lipofectamine2000 mismatch siRNA, LG: lipofectamine2000 with targeted GFP siRNA, CM: Chitosan mismatch siRNA, CG: chitosan targeted GFP siRNA.

The present invention will now be described in more detail in the following.

### DETAILED DESCRIPTION OF THE INVENTION

It is an object of the present invention to provide a scaffold with optimal mechanical properties and controlled API-release profile for a therapeutic strategy based on tissue engineering.

One aspect of the invention relates to a three-dimensional hybrid scaffold capable of supporting cell activities such as growth and differentiation, and capable of controlled release of active pharmaceutical ingredients, characterized in that the scaffold comprises a first and a second biocompatible material, said first material shaped as a framework forming one or more open networks of voids, said second material comprising an ion exchange material, said ion exchange material being loaded with one or more active pharmaceutical ingredients.

The term “hybrid scaffold” is to be understood as the first biocompatible material and the second biocompatible material having different functions. The first biocompatible material mainly functions to support cell activities such as growth and differentiation, whereas the second biocompatible material mainly functions to control the release of active pharmaceutical ingredients.

The term “active pharmaceutical ingredient (API)” refers to the biologically active substance in a pharmaceutical drug, such as e.g. an anticancer drug, where the API is doxorubicin.

The term “controlled release” is also known as sustained-release, sustained-action, extended-release, time-release or timed-release, modified release, or continuous-release, and refers to a mechanism used to release a drug/API slowly over time. One advantage of controlled release is that it keeps steadier levels of the drug in the bloodstream or at a specific site. Doxorubicin, a well-known anticancer drug, is an anthracycline antibiotic with a broad spectrum antitumor activity to treat several types of cancer. However, treatment with doxorubicin is restricted due to several undesirable side effects like its cumulative-dose limit and cardiotoxicity (Kumar, Kirshenbaum et al. 2001; Takemura and Fujiwara 2007). Hence, in one embodiment of the present invention, the ion exchange material is loaded with anticancer drugs, such as doxorubicin.

The term “anticancer drug” in the present invention refers to a drug used in cancer therapy. Concrete examples of the anticancer drug include a cytotoxic anticancer drug, a molecular target-based drug, hormone and the like.

The term “cytotoxic anticancer drug” refers to an anticancer drug which demonstrates any influence on the DNA of a nucleus or the cell cycle to exert the anticancer effect. Concrete examples of the cytotoxic anticancer drug include an alkylating agent, an antimetabolite, an anticancer antibiotic, a platinum-based drug and a plant alkaloid and the like.

The alklyating agent includes nitrogen mustard N-oxide hydrochloride, melphalan, cyclophosphamide, ifosfamide and other nitrogen mustard; carboquone, thiotepa and other ethyleneimines; nimustine hydrochloride, ranimustine and other nitrosoureas and the like.

The antimetabolite includes methotrexate and other folic acid antagonists; fluorouracil, tegafur, carmofur and other pyrimidine antagonists; cytarabine, cyclocytidine, enocitabine and other cytosine arabinoside; mercaptopurine, thioinosine and other purine antagonists and the like.

The anticancer antibiotic includes adriamycin, doxorubicin, daunorubicin, aclarubicin, pirarubicin and other anthracyclines; bleomycin, pepleomycin and other bleomycins; mitomycin C and other mitomycins actinomycin D and other actinomycins; neocarzinostatin and other polypeptides and the like.

The platinum based drug includes cisplatin, carboplatin, nedaplatin, oxaliplatin and the like.

The plant alkaloid includes vinblastine, vincristine, vindesine, paclitaxel, docetaxel, etoposide, camptothecine, irinotecan and the like.

Topoisomerases are popular targets for cancer chemotherapy treatments. Topoisomerase inhibitors are agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle. Topoisomerase inhibitors are divided according to which type of enzyme it inhibits. Examples of topoisomerase I inhibitors are irinotecan, topotecan, camptothecin and lamellarin D all target type IB topoisomerases. Examples of topoisomerase II inhibitors are amsacrine, etoposide, etoposide phosphate, teniposide and doxorubicin.

In principle, every API suitable for controlled release is applicable in the present invention.

Macroporous polycaprolactone (PCL) scaffolds made by fused deposition modelling (FDM) have potential as ideal base scaffolds for bone tissue engineering because of their advantageous mechanical and biodegradable properties (Hutmacher, Sittinger et al. 2004). Hence, in one embodiment of the present invention, the first biocompatible material shaped as a framework forming one or more open networks of voids comprises polycaprolactone (PCL).

Chitosan-tricalcium phosphate (TCP) foam can improve a scaffolds' osteoconductive property and increase the scaffolds' surface area (Lee, Park et al. 2000). In another embodiment of the present invention, the second biocompatible material comprises chitosan-tricalcium phosphate (TCP). In yet another embodiment of the present invention, the second biocompatible material is in the form of porous foam.

The term “ion exchange material” refers to both an anion exchange material and a cation exchange material. As used herein, the term “anion exchange material” means a material which is preferentially conductive to anionic species. In this respect, such material is configured to selectively exchange anionic species present in the material, such as an API, for anionic species from the surroundings. As used herein, the term “cation exchange material” means a material which is preferentially conductive to cationic species. In this respect, such material is configured to selectively exchange cationic species present in the material, such as an API, for cationic species from surroundings. Non-limiting examples of ion exchange materials are clay, such as a clay mineral derivative, ion exchange polymers, ion exchange resins, layered double hydroxides, and zeolites.

In one embodiment of the present invention, the ion exchange material is selected from the group consisting of clay, clay mineral derivatives, ion exchange polymers, ion exchange resins, layered double hydroxides, zeolites and mixtures thereof.

In one embodiment of the present invention, the ion exchange material is a Layered Double Hydroxide (LDH) derivative.

Layered double hydroxides (LDH) comprise a class of layered materials with positively charged layers and charge balancing anions located in the interlayer region. In one embodiment of the present invention, the layered double hydroxide has a layered structure corresponding to the general formula:

[M<2+>1−xN<3+>x(OH)2][A<n−>]x/n*yH2O,

wherein M<2+> is a divalent metal ion such as Zn²⁺, Mn²⁺, Ni²⁺, Co²⁺, Fe²⁺, Cu²⁺, Sn²⁺, Ba²⁺, Ca²⁺, and Mg²⁺, most preferably Mg²⁺; M<3+> is a trivalent metal ion such as Al³⁺, Cr³⁺, Fe³⁺, Co³⁺, Mn³⁺, Ni³⁺, Ce³⁺, and Ga³⁺, most preferably Al³⁺; x is 0.1 to 0.4, A is an anionic API, n is a charge number of the API, and y is a positive number.

In the above formula, x relates to a metal composition ratio and may range from 0.1 to 0.4, and more preferably from 0.25 to 0.33. If the x value is outside of this range, the intercalation of an API between the hydroxide layers of the LDH carrier may not occur, which renders the production of a desired API-LDH hybrid difficult.

The API-LDH hybrid of the present invention may be used in a hydrate form. The 5 degree of hydration can be expressed as the y value. The y value can be changed according to various factors such as moisture content in air, and can be represented by a positive number, generally selected within a broad range.

In another embodiment of the present invention, A is a charge-balancing anion or a mixture of charge-balancing anions. Examples of suitable charge-balancing anions are carbonate, hydroxyl, nitrate, chloride, bromide, sulfate, bisulfate, phosphate, organic anions, and combinations thereof. Hydroxyl, carbonate and organic anions are preferred. Because the LDH has a layered structure, the charge-balancing anions may be situated in the interlayer, on the edges, or on the outer surface of the stacked LDH layers. The anions situated in the interlayer of stacked LDH layers are referred to as intercalating anions. LDHs containing charge-balancing organic anions are rendered organophilic and are also referred to as “organoclays”. In still another embodiment of the present invention, A is a mixture of one or more charge-balancing anions and one or more API's.

In still another embodiment of the present invention, the Layered Double Hydroxide (LDH) derivative further comprises an intercalating and/or exfoliating biocompatible or biodegradable polymer, such as e.g. chitosan, alginate, gelatin, and polyethylene glycol.

In another embodiment of the present invention, the ion exchange material is a clay mineral derivative. In a specific embodiment of the present invention, the clay mineral derivative is selected from the group of Phyllosilicate derivatives; in particular 2:1 phyllosilicates made of two tetrahedral layers and one octahedral layer, and 1:1 phyllosilicates made on one tetrahedral layer and one octahedral layer. Non-limiting examples of 2:1 phyllosilicate derivatives are smectite, vermiculite, mica, chlorites, and non-limiting examples of 1:1 phyllosilicate derivatives are kaolin and serpentine. The smectite group comprises non-limiting examples such as saponite, hectorite, sauconite, montmorillonite, beidellite, nontronite, stevensite. The vermiculite group comprises non-limiting examples such as trioctahedral vermiculite and dioctrahedral vermiculite. The mica group comprises non-limiting examples such as phlogopite, biotite, lepidlite, muscovite, paragonite, chlorite, margarite, taeniolite, and tetrasilicic micac. The phyllosilicates may be natural products, or synthetic ones produced e.g. by the hydrothermal method, melting method or the solid phase method.

In one embodiment of the present invention, the clay mineral derivative further comprises an intercalating and/or exfoliating biocompatible polymer, such as e.g. chitosan, alginate, gelatin, and polyethylene glycol.

In another embodiment of the present invention, the three-dimensional hybrid scaffold further comprises a binder.

The term “binder” refers to a material capable of binding together two or more materials in the three-dimensional hybrid scaffold, such as the first and the second biocompatible material. The binder's principal properties are adhesion and cohesion. In one embodiment of the present invention, the binder comprises at least one synthetic or naturally derived biocompatible polymer or a synthetic or naturally derived hydrogel or combinations thereof.

The term “biocompatible” is to be understood as eliciting little or no immune response in a given organism. Indeed, since the immune response and repair functions in the body are highly complicated, it is not adequate to describe the biocompatibility of a single material in relation to a single cell type or tissue.

In one embodiment of the present invention, the three-dimensional hybrid scaffold is biodegradable. The term “biodegradable” is to be understood as, but not limited to, the chemical, such as biochemical, breakdown of materials by a physiological environment, such as in a human or animal.

The term “open networks of voids” is to be understood as, but not limited to, a delimited hollow space within the walls or surfaces of the first biocompatible material.

In one embodiment of the present invention, the first biocompatible material is shaped as one or more grids of nano to micron sized strands.

In yet another embodiment of the present invention, the second material is filling 0.001-100% of the total void volume of said open networks, such as in the range of 0.005-95%, e.g. 0.01%, such as in the range of 0.015-90%, e.g. 0.02%, such as in the range of 0.05-85%, e.g. 0.1%, such as in the range of 0.2-75%, e.g. 0.5%, such as in the range of 1-65%, e.g. 3%, such as in the range of 5-55%, e.g. 10%, such as in the range of 0.15-45%, e.g. 20%.

The term “scaffold” is to be understood as, but not limited to, the construct ready for implantation, e.g. suitable for use as an implant. In certain aspects of the invention, the scaffold is cut before implantation to fit the defect.

The size of the scaffold must reflect the defect size, but also the migration rate of cells, e.g. within the scaffold, should be taken into account.

In another embodiment of the present invention, the second biocompatible material is in the form of a coating layer.

In yet another embodiment of the present invention, the second biocompatible material is in the form of a paste.

In still another embodiment of the present invention, the second biocompatible material is in the form of a foam.

In one embodiment of the present invention, the scaffold has a comparable value of compression stress to the value of compression stress of the targeted tissue.

In still another embodiment of the present invention, the framework has a value of initial compression stress of at least 3 kPa. In one embodiment of the present invention, the scaffold has a comparable value of compression stress to the value of compression stress of the targeted tissue. As an example, the inventors have produced a scaffold with a measured value of compression stress of 5100 kPa. Such a scaffold could be useful for trabecular bone reconstruction, where the trabecular bone has a value of compression stress of about 5000 kPa. A “comparable” value is to be understood as a value not deviating more than 35% from the other value, such as 25%, e.g. 20%, preferably not deviating more than 10%, e.g. 5%.

In a preferred embodiment of the present invention, the compression stress of the framework is 5 times higher than the second biocompatible material, such as in the range of 5-100000 times higher, e.g. 10 times higher, such as in the range of 15-90000 times higher, e.g. 50 times higher, such as in the range of 55-90000 times higher, e.g. 100 times higher, such as in the range of 200-50000 times higher, e.g. 500 times higher, such as in the range of 700-30000 times higher, e.g. 900 times higher, such as in the range of 1.500-20.000 times higher than the second biocompatible material.

In one embodiment of the present invention, the three-dimensional hybrid scaffold surface is coated with a natural or synthetic coating material.

In one embodiment of the present invention, the scaffold surface is coated with a natural or synthetic coating material such as protein, peptides, nucleotides, and/or small interfering RNAs.

In another embodiment of the present invention, the scaffold surface is coated with a material selected from the group consisting of proteins, peptides, nucleotides, and small interfering RNAs or mixtures thereof.

In one embodiment of the present invention, the three-dimensional hybrid scaffold further comprises apatites or mixtures thereof. Apatite is a group of phosphate minerals, usually referring to hydroxyapatite, fluorapatite, chlorapatite and bromapatite, named for high concentrations of OH⁻, F⁻, Cl⁻ or Br⁻ ions, respectively, in the crystal. The formula of the admixture of the four most common members is written as Ca₁₀(PO₄)₆(OH, F, Cl, Br)₂.

Yet another aspect of the present invention relates to the use of the engineered three-dimensional hybrid scaffold for soft tissue repair or regeneration.

Still another aspect of the present invention relates to the use of the engineered three-dimensional hybrid scaffold for brain or spinal cord tissue repair or regeneration.

Another aspect of the present invention relates to the use of the three-dimensional hybrid scaffold in the repair or regeneration of a tissue selected from the group consisting of bone, soft tissue, cartilage, brain and spinal cord tissue.

Another aspect of the present invention relates to the engineered three-dimensional hybrid scaffold for use as a medicament. Preferably, the three-dimensional hybrid scaffold is biodegradable.

Yet another aspect of the present invention relates to the tissue engineering of complete or parts of organs (e.g. liver, kidney, and lung), such as the engineered three-dimensional hybrid scaffold for use in the treatment of liver diseases, kidney diseases, lung diseases, bone diseases, cartilage diseases or other tissue diseases.

Another aspect of the present invention relates to the use of the engineered three-dimensional hybrid scaffold for bone repair or regeneration, such as the engineered three-dimensional hybrid scaffold for use in the repair or regeneration of brain or spinal cord tissue.

Yet another aspect of the present invention relates to the use of the engineered three-dimensional hybrid scaffold for cartilage tissue repair or regeneration.

Another aspect of the present invention relates to the three-dimensional hybrid scaffold functioning to release birth control agents continuously over time and does not have to be removed by surgery.

In one embodiment of the present invention, the process for manufacturing the three-dimensional hybrid scaffold is performed by incorporating the second biocompatible material into the framework either by simple wet compaction or, preferably, by mixing it with a binder. The clay matrix can be clay mineral (such as montmorrilonite), layered double hydroxides (LDH) (such as hydrotalcite), organoclay or mixtures thereof. The binder comprises at least one synthetic or naturally derived biocompatible polymer or a synthetic or naturally derived hydrogel or combinations thereof.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

The objective of this study was to develop a biocompatible implant that would be placed at the defect site during tumor resection surgery. This construct would provide adequate structural (mechanical) support, locally, provide a controlled sustained release of a chemotherapeutic agent, and eventually promote tissue ingrowth.

### Materials and Methods

### Materials

The nanoclay used in this study was from Southern Clay Products, Inc., Germany (Cloisite Na+, Lot: 07F28GDX-008). Chitosan (Chitopharm M) with 75-85% degree of deacetylation was obtained from Cognis. Polycaprolactone (MW=50 kDa) was from Perstorp, UK. Beta-tricalciumphosphate nanocrystals (TCP, Lot: TCPCH01) was purchased from Berkeley advanced bimaterials, Inc., USA. Doxorubicin hydrochloride (DOX) was from Sigma, Denmark.

### Scaffold Fabrication

### PCL-Base Scaffold Manufacture

Scaffolds were made from PCL by fused deposition modeling with a BioScaffolder (SYS+ENG GmbH, Germany). Cylindrical scaffolds with a diameter of 10 mm were punched out from a 5 mm thick porous PCL mats with a biopsy punch (Acuderm, Florida). To increase surface hydrophilicity and thus improve cell attachment, the scaffolds were etched in 5 M sodium hydroxide for 3 hours, and then 70% ethanol for sterilization treatment. The scaffolds were rinsed in sterile water multiple times and dried.

### Clay Modification

A pilot study showed that the clay-DOX carrier released less than 10% in 1 month. Therefore, the clay was tried modified with chitosan.

The clay was modified with chitosan as described by Yuan, Shah et al. 2010. The clay was briefly dispersed in 0.2% (w/v) chitosan solution in 1.0% (v/v) acetic acid to a chitosan/clay weight ratio of 10:1. After stirring for 4 hrs at about 500 rpm, the colloidal suspension was washed with 1.0% (v/v) acetic acid three times to remove free chitosan. Finally, the modified clay nanoparticles pellet was dispersed in 1.0% (v/v) acetic acid and ready for scaffold fabrication.

### Modified Clay/DOX Carrier

The modified clay was dispersed in DOX solution for 12 hrs and vortex for 2 hrs. Then the solution was centrifuged at 15,000 g for 10 minutes and the supernatant was collected. DOX was encapsulated into the clay nanoparticles and named as modified clay/DOX carrier.

### Preparation of Composite Scaffolds

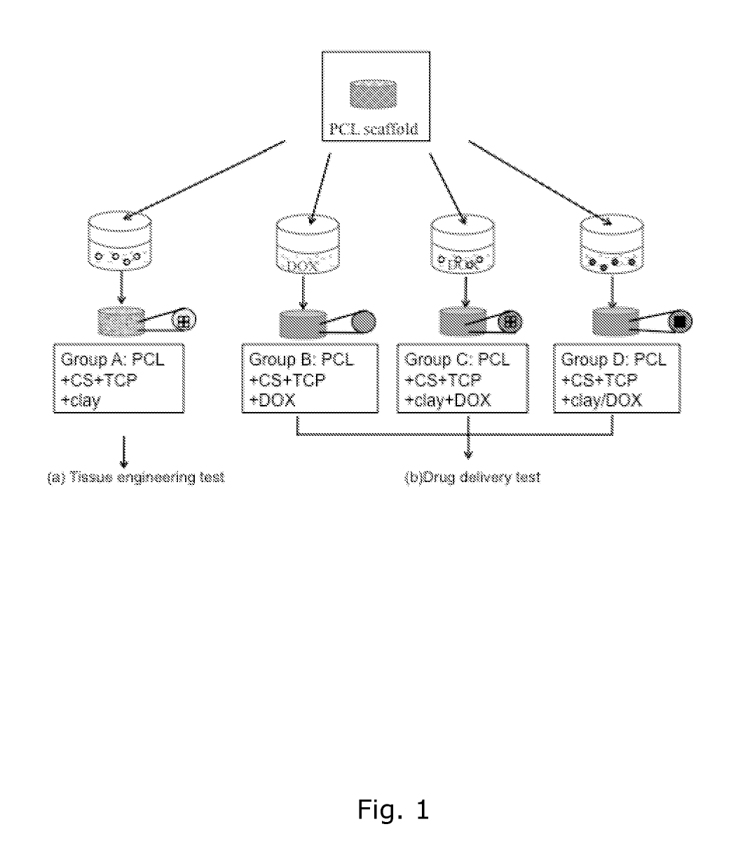

TCP nanoparticles were dispersed in 1% w/v chitosan in 1 volume % acetic acid solution to a TCP/chitosan ratio of 1:20. The chitosan-TCP solution was stirred at room temperature and then divided into four groups: A, B, C, and D (FIG. 1). Group A was added with clay nanoparticles, as a blank scaffold, for the bone tissue engineering test. Group B was added with DOX, as a control group for the drug delivery test. Group C was added with both clay and DOX, and group D added with modified clay/DOX carrier. Groups C and D were the testing groups for drug delivery. PCL scaffolds were immersed into 500 μl of four different solutions, and then scaffolds were freeze-dried (FTS, NJ, US). The combinations of each scaffold are shown in Table 1.

TABLE 1
Scaffold composition in different groups.

| | Group | | | | |
|---|---|---|---|---|---|
| per scaffold | A | B | C | D | Unit |
| 1% chitosan | 500 | 500 | 500 | 500 | μl |
| TCP | 0.25 | 0.25 | 0.25 | 0.25 | mg |
| clay | 0.45 | / | 0.45 | / | mg |
| DOX | / | 0.1 | 0.1 | / | mg |
| Clay/DOX | / | / | / | 0.45/0.1 | mg |

### Drug Release Profile Test

The release profile of DOX from the scaffolds was determined (monitored) by incubating a scaffold in 1.0 ml of PBS (pH=7.4) at 37° C. (n=4) for 2 months. At each time point, 1 ml of solution was collected and replaced with 1 ml of fresh PBS. The fluorescence intensity of DOX in the buffer solution was quantified with a Victor 1420 multilabel counter (Wallac, USA) with excitation at 405 nm and emission at 615 nm. The concentrations of DOX released in the solutions were calculated according to the calibration curve of DOX in PBS.

Seeding hMSC-TERT Cells to Scaffold

A telomerase reverse transcriptase gene-transduced cell population, hMSC-TERT cells, was used in this study. These cells maintain the functional characteristics of primary MSCs and have the capability to differentiate into certain mesodermal cell types (osteoblasts, chondrocytes, and adipocytes) upon specific stimuli (Simonsen et al., 2002). Cells from PD level 262 (passage 45) were seeded at a density of 4000 cells/cm² in culture flasks in Dulbecco's modified essential medium (DMEM) containing 10% fetal bovine serum (FBS) and cultivated in a humidified atmosphere of 37° C. and 5% CO₂.

After one week, cells were washed in PBS, detached with 0.125% trypsin and 5 mM EDTA in PBS, reseeded, and cultured for another week. Cells were trypsinized (PD level 271, passage 47) and resuspended for use (2×10⁷ cells/ml) in DMEM/10% FBS (penicillin (100 U/ml) and streptomycin (100 mg/l). hMSC-TERT cells were seeded onto the scaffolds by pipetting 50 μl of cell suspension media with 1×10⁶ cells onto each scaffold. The scaffolds were placed in agarose-coated (1% in sterile H₂O) six-well plates (4 scaffolds/well), and incubated for 2 hours in an incubator. Thereafter, additional 7.5 ml of DMEM/10% FBS, 100 U/ml penicillin, 100 mg/l streptomycin were added to each well. After 24 hours, cell/scaffold constructs were moved to 58 mm diameter dual side-arm spinner flasks (Bellco glass, Vineland N.J., USA). An autoclavable stainless framework with four needles was constructed and placed in the spinner flasks. Two cell-seeded scaffolds were mounted on each needle giving a total of eight scaffolds per flask. Spinner flasks containing 120 ml of media each were placed on a Bell-ennium™ five position magnetic stirrer (Bellco Biotechnology, NJ, USA) at 30 revolutions per minute in the incubator with side arm caps loosely attached. Cell/scaffold constructs were cultured with DMEM/10% FBS for the first week, and then medium was replaced with osteogenic stimulation medium (DMEM/10% FBS with 100 nM dexamethasone, 290 μM ascorbic acid and 5 mM β-glycerophosphate) (all from Sigma) and cultured for up to 21 days. Media was exchanged twice a week.

Cellular Adhesion, Viability and Proliferation of hMSC-TERT Cellular Scaffolds

### Scanning Electron Microscope (SEM)

Scaffolds were rinsed in PBS and fixed in 2.5% glutaraldehyde containing 0.1M sodium cacodylate buffer (pH 7.4) and dehydrated in a graded ethanol series, air-dried. The samples were viewed using environmental mode SEM (Nova NanoSEM 600, FEI Company) and element component of crystal-like structure was analyzed by energy dispersive X-ray spectrometer (EDX).

### Confocal Imaging

To assess cell viability, the cell/scaffold constructs were incubated for 30 minutes in DMEM containing 10 μM CellTracker™ Green CMFDA (Invitrogen, Denmark). The staining medium were then replaced with fresh DMEM/10% FBS and incubated for another 30 min at 37° C. Non-fluorescent CMFDA is converted to brightly green fluorescent product when cytosolic esterases cleave off the acetates. The cell/scaffold constructs were then rinsed in prewarmed PBS, fixed in 10% formalin for 5 min at room temperature, and stained with 1 μg/ml Hoechst 33258 (Sigma-Aldrich, Denmark) in PBS for 20 min. Living cells were labelled with green pixels. Nuclei of the cells were stained with Hoechst, labelled with red pixels. Chitosan were stained with yellow pixels resulting from the spatial overlap of red and green pixels. Images were acquired using a laser scanning confocal microscope, 510 Meta (Zeiss Microimaging GmbH, Germany). The confocal settings (excitation, laser power, detector gain, and pinhole size) were same for all cell imaging. Separate channels and filters were used. Excitation/emission wavelengths were 488 nm/505-530 nm for CellTracker™ Green and 405 nm/420 nm for Hoechst.

### DNA Quantification

The total cell number in the 3D cellular scaffold was estimated by quantifying the dsDNA content in each scaffold using the Quant-iT™ PicoGreen® dsDNA assay (Invitrogen). Scaffolds were thawed, and sonicated in intervals of 1 seconds on/5 seconds off for a total of 1 min. 3 mg of collagenase (Sigma, Denmark) were added to each DNA sample and the samples were incubated in a 37° C. water bath for 3 h. 1 mg proteinase K (Sigma, Denmark) was then added and the samples were incubated overnight in a 45° C. water bath. Sample volume was diluted 1:10 in Tris-EDTA buffer and vortexed to release DNA from scaffold debris. From each sample, 2×50 μl were drawn, 50 μl of PicoGreen (diluted 1:200 in TE buffer) was added, incubated for 5 min in the dark, and measured in a 96-well plate using a microplate reader, Victor3 1420 Multilabel Counter, (PerkinElmer Life Sciences, Denmark). Samples were excited at 480 nm, and the fluorescence emission intensity was measured at 520 nm. Standards were prepared according to manufacturer's instruction (lambda DNA, concentration range: 0-1 μg/ml). Technical duplicates were used for each biological sample (n=4).

Osteogenic Differentiation and Mineralization of hMSC-TERT Cells in 3D Scaffold

### Alkaline Phosphatase (ALP) Activity Assay

ALP activity was determined using a colorimetric endpoint assay measuring the enzymatic conversion of p-nitrophenyl phosphate (Sigma, Denmark) to the yellowish product p-nitrophenol in the presence of ALP. Absorbance of p-nitrophenol was measured by microspectrophotometer (Victor 1420, Perkin Elmer, Denmark) at double wavelengths of 405 nm and 600 nm. Standards were prepared from p-nitrophenol (concentration range: 0-0.2 mM). Technical duplicates were used for each biological sample (n=4).

### Von Kossa Staining

Scaffolds were gently rinsed with PBS and fixed for five min in 4% (w/v) formaldehyde solution (pH 7.0), then washed with ddH₂O, incubated with a 2.5% silver nitrate solution for 20 min in the dark, and subsequently developed by adding 0.5% hydroquinone for two min. Finally, surplus silver was removed using sodium thiosulphate for five min. Scaffolds were dried under vacuum and pictures taken.

### Calcium Content Assay

Calcium contents of cell-seeded scaffolds were quantified by colorimetric endpoint assay based on the complexation of one Ca²⁺ ion with two Arsenazo III molecules to a blue-purple product (Diagnostic Chemicals Limited, Charlottetown, PE, Canada). Briefly, the calcium deposition is dissolved in 1M acetic acid by placing in a shaker overnight. The samples were diluted 1:50 with ddH2O and aliquots of 20 μl were transferred to a 96-well plate. Arsenazo III solution (280 ml) was added and incubated for 10 min at room temperature. A standard dilution series of calcium ranging from 0 to 50 μg/ml was prepared and Ca²⁺ concentration was quantified spectrophotometrically at 650 nm. Calcium content was expressed as micrograms of Ca²⁺ per scaffold.

### Histology and Immunohistochemistry

Scaffolds were fixed in 70% ethanol, Technovit® 7100 (Ax-lab, Vædbk, Denmark) embedded, and cut in 25 μm sections using a Sawing Microtome KDG 95 (Meprotech, the Netherlands). Sections were taken from the peripheral and the central part of the scaffold. H&E staining was applied to reveal the cell distribution. Histochemical staining for ALP was performed to test the osteogenic phenotype of cells cultured in the scaffolds. For immunohistochemistry, the sections were incubated overnight with the rabbit anti-human osteocalcin antibody (Biomedical technologies Inc, BT593), followed by a biotinylated goat anti-rabbit IgG (DAKO E0432) for 1 hour, and peroxidase-conjugated streptavidin (DAKO PO397) for 1 hour. Sections were visualized with 3-amino-9-ethylcarbazol (Sigma A6926) and counter-stained with Mayer's hematoxilin. Images were photographed using a BX50 microscope with a Camedia C-5060 camera (Olympus, Denmark).

### Statistical Analysis

Results are presented as mean±standard deviation (SD) for n=4 biological replicates. Statistics were assessed using Stata 10.0 (College station, TX). The data of DNA quantification, ALP activity, and calcium content were determined using one-way ANOVA.

Differences between means were considered statistically significant when p-values <0.05.

### Results

Drug Release from Scaffolds

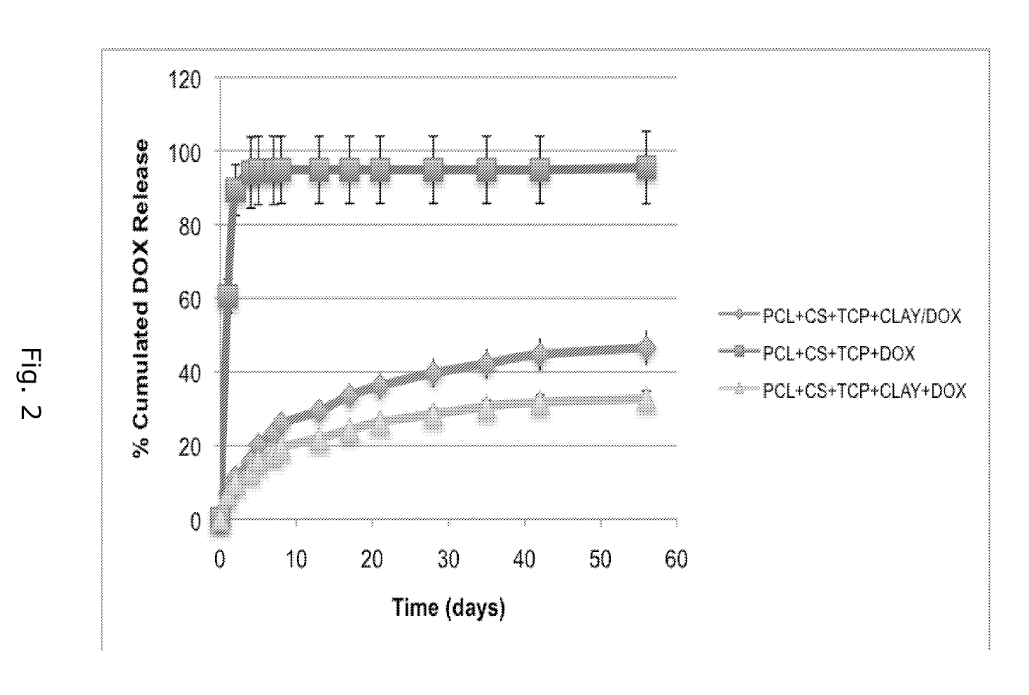

DOX alone, together with clay, or modified clay/DOX carrier was incorporated into scaffolds. The release profile of DOX from these three different composite scaffolds is depicted (described) in FIG. 2. There was an initial burst release in all the groups. On day 4, DOX release was (94.1±9.6) % of the total amount of drug in the control scaffolds (Group B, pure DOX without clay). Compared with the clay incorporated scaffolds, only (13.0±0.2) % of DOX was released from Group C (DOX and clay mixed solution) and (15.7±1.0) % was released from Group D (modified clay/DOX carrier). The cumulative release was statistically lower from Group C than Group D from day 5 (p=0.04). By day 56, about 32.8% was released from Group C and 46.6% released from Group D.

### Cell Adhesion, Viability and Proliferation in the Scaffold

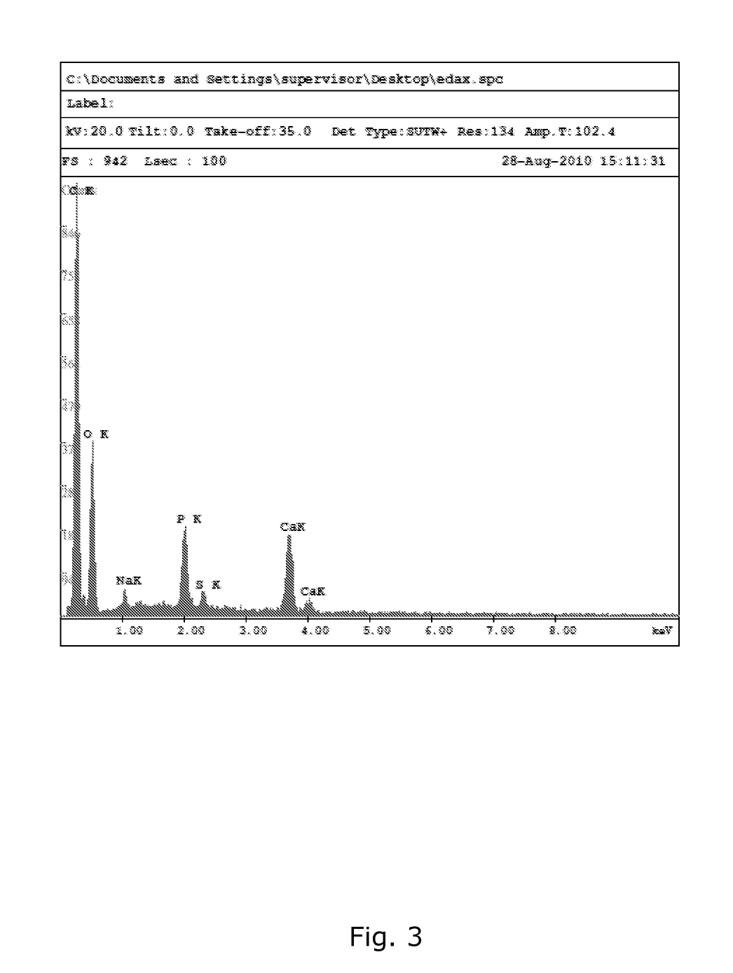

Scanning electron microscopy (not depicted) showed the cells and extracellular matrix deposition on the scaffolds. On day 1, the cells anchored tightly on the surface of the scaffold. Cells were at the status of attachment and spreading on the scaffolds. On day 7, cells and extracellular matrix deposition partially covered the scaffolds. Increasing density and extracellular matrix deposition almost completely covered the scaffolds on day 14. Crystal-like extracellular matrix deposition was observed on the surface of the scaffolds with 21-days culture. These deposits were expected to be calcium phosphate salts which was identified by element component analysis (EDX) to consist of mainly P, Ca, and O (FIG. 3).

Confocal microscopy images (not depicted) showed the good cell viability in scaffolds during the 21 days culture. Cells attached and spread in the scaffolds from day 1. Cells migrated into the macro- and micropores of the scaffolds and spread evenly on the surface of scaffolds. Cell density increased steadily with culture days. Higher magnification showed the cells grew into the chitosan structure and rapidly proliferated (the nuclear density increased).

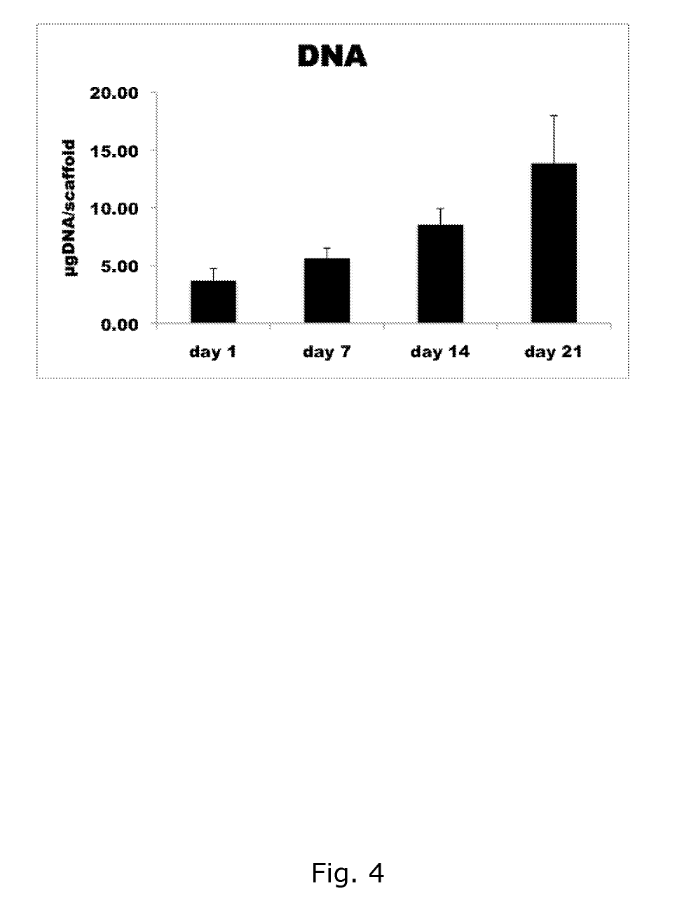

The DNA amount was assumed proportional to the cell number. Thus, cell proliferation over time could be followed by quantification the extracted DNA from the scaffolds. DNA amounts were increasing during the culture period (FIG. 4).

Osteogenic Differentiation and Mineralization of hMSC-TERT Cells in 3D Scaffold

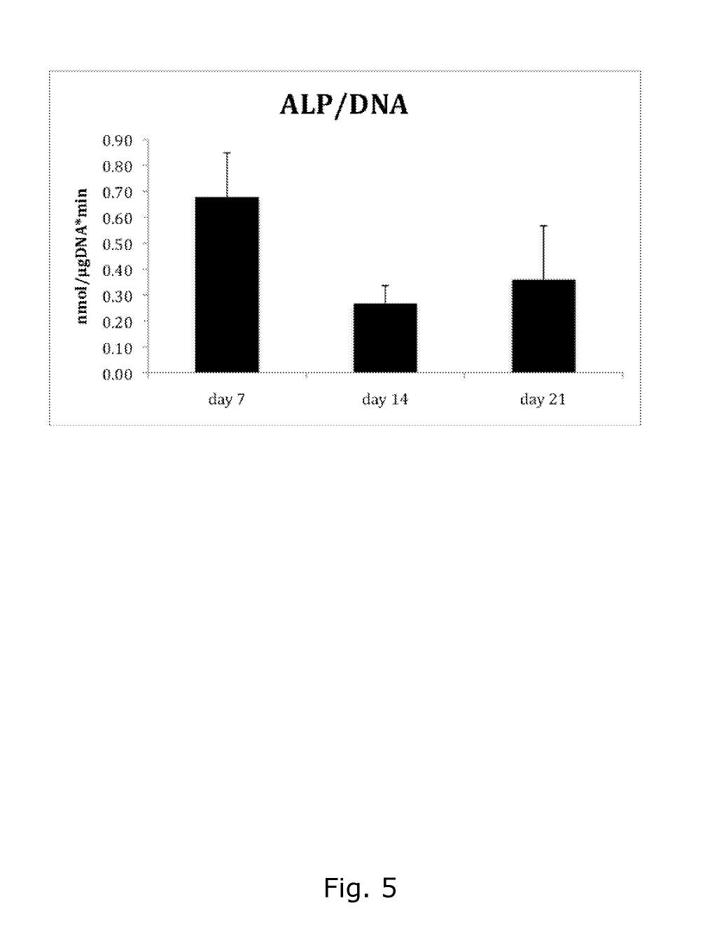

ALP activity was highest on day 7 and then decreased to day 14, and then maintained the same level until day 21. This suggested that the cells start to initiate mineralization on day 7 (FIG. 5).

ALP positive staining (brown colour) confirmed the presence of ALP, which was a component and marker for the extracellular matrix produced by osteogenic differentiated cells (Not depicted).

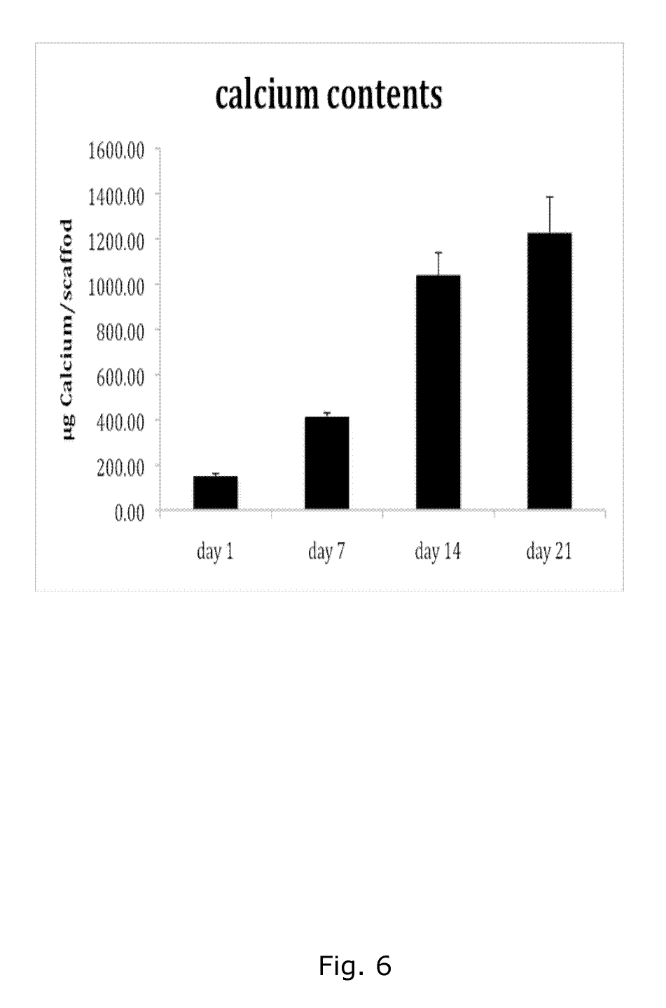

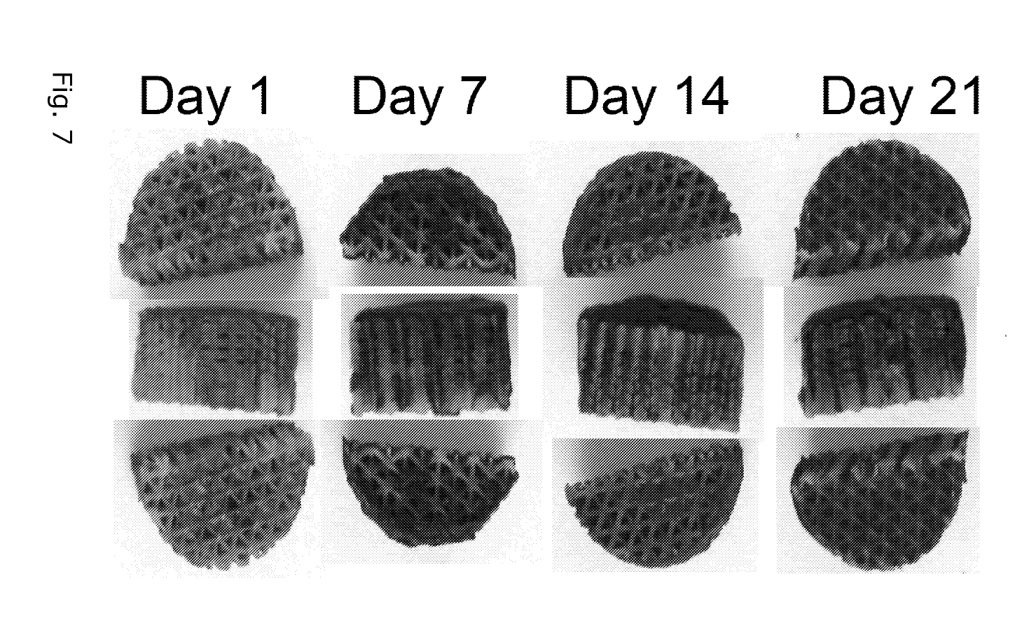

Quantitative data of calcium content (FIG. 6) and von Kossa staining (FIG. 7) showed that the scaffolds were osteoconductive.

### Histology

Cross-sections of the scaffold with H&E staining revealed the cellular distribution within the scaffold (not depicted). Cells migrated into the center of the scaffolds within 7 days of culture and the pores of the scaffolds were partly filled with cells and new extracellular matrix. The depth of cell infiltration and the density of the cells increased with culture time. On day 21, cells had fully filled the macro- and micropores of the scaffolds.

The positive osteocalcin staining (red dots, pointed with arrows) showed that hMSCs-TERT cells were osteogenic differentiation and secreted bone related extracellular matrix marker (not depicted).

### Discussion

We succeeded in fabricating a hybrid scaffold consisting of chitosan/nanoclay/polycaprolactone/β-tricalcium phosphate, which served as a mechanically stable biocompatible bone tissue engineering scaffold, as well as a local sustained drug release system.

Tumor resection is followed by systemically administered chemotherapy to prevent the tumor recurrence. The limitations of systemic drug administration include the diluted drug concentration at the targeted cancer cells, the systemic toxicity, and adverse effects. Systemically administered, targeted drug delivery has gained increased interests with the development of nanotechnology and biomaterials. Current efforts have been focused on developing drug-nanoparticles, such as drug-incorporated liposomes and particulate drug-polymer carriers. Controlled drug release by directly delivered to the tumor site is at present an approach far easier to implement. The Gliadel® Wafer, which is used against brain tumors, is one early example of a local drug delivery system in clinical use. Other local drug delivery systems for other tumor types are being researched. Itokazu, Sugiyama et al. (1998) showed that methotrexate released from porous hydroxyapatite blocks and 8-TCP blocks remained effective against tumor cells up to 12 days in vitro. Fröschle, Mählitz et al. (1997) reported that daunorubicin-polymethylmethacrylate filled in the resection cavity delayed or reduced the number of recurrences in bone metastasis animals. EI-Ghannam, Ricci et al. (2010) tested a ceramic-based anticancer drug, 5-Fluorouracil, to treat breast cancer in a murine model. However, the drug release patterns were difficult to control in those systems and initiate burst release was often reported.

In the present study, it is shown that DOX was controlled released from the clay loaded composite scaffolds for two months. Only about 50% DOX was released from the clay-loaded scaffolds, while 100% DOX was released from the non-clay loaded scaffolds on day 4. This shows that clay is an effective material to modulate drug delivery and control drug release.

Additionally, the present study showed that DOX controlled release was tunable with fabrication procedure. With the same composites, the modified clay/DOX carrier group had a faster release compared to the group that DOX and clay were directly added. This may be due to the clay, drug, and chitosan composited different structures. When DOX and clay were directly added into the chitosan solution, DOX could intercalate into clay layers by ion exchange, and formed intercalation when mixed with chitosan solution. In the other group, modified clay/DOX carrier was prepared with 2 hours votex. This shear might delaminate some of the silicate layers of the clay and formed exfoliation when mixed with chitosan solution, so that DOX released faster. Different fabrication procedure could tune the release kinetics of the drugs. The ratios of the composites could do the same. It is contemplated that drug release kinetics could be adjusted by altering clay/chitosan/drug ratios in the composite scaffolds of the present invention.

In the special case of bone cancer, patients need bone grafts or artificial grafts to be placed at the resected tissue site to provide immediate mechanical support. In this study, PCL by FDM was used as a base scaffold to offer an adequate mechanical property. Furthermore, a micro porous foam consisting of chitosan/nanoclay/β-TCP was added to the base PCL scaffold to obtain a better biocompatibility and osteogenesis. It was shown that hMSCs-TERT cells had high cell viability and grew into the macro and micro pores of the scaffolds, confirmed by SEM, confocal microscopy, and histology. Calcium phosphate, like β-TCP, and hydroxyapatite is widely applied as coating on other implants like titanium to achieve an earlier and greater bone ingrowth (Lickorish, Ramshaw et al. 2004; Taché, Gan et al. 2004). Chitosan, a fascinating natural polymer, has been widely investigated to be used in the tissue engineering and drug delivery fields along with favourable biological properties including biocompatibility, biodegradability, nontoxicity, fungistatic and antibacterial properties (Di Martino, Sittinger et al. 2005). Combining the advantages from these different biomaterials, hMSCs-TERT cultured in these composites were osteogenic differentiated and mineralized.

Therefore, our composite scaffold can be used as a bone tissue-engineering substitute, as well as a drug delivery system. Furthermore, in vivo studies on this composite scaffold are underway.

Due to the unique property of clay minerals, different kinds of drugs can be loaded into this system to have different therapeutically aims. The drug loaded efficiency and drug release kinetics are tunable since the ratios of the components can be adjusted.

### CONCLUSION

The inventors of the present invention have fabricated a 3D hybrid scaffold, that comprises a porous foam comprising a chitosan/clay/β-TCP embedded in a PCL-based scaffold. This composite scaffold has an adequate mechanical property owing to the PCL-based scaffold, and offers a favourable environment for cell attachment, proliferation and osteogenic differentiation. This novel composite porous scaffold material has the potential for a wide range of bone tissue engineering.

Besides as a bone tissue engineering substitute, this composite scaffold can also be loaded with anticancer drugs like doxorubicin functioning as a local drug delivery system to prevent cancer recurrence. By changing the PCL-based scaffold with other adequate mechanical property based scaffolds, this drug-loaded system can be investigated for other solid tumors like breast cancer treatment.

### Drug Loading and Release Tests

In this study, the above composite scaffold was used for eluting different drugs (anticancer drug Doxorubicin and two model proteins, BSA and Lysozyme). Scaffolds were prepared with the same formula as Group A, as described above. Doxorubicin, BSA (Bovine Serum Albumin) or Lysozyme was loaded into the scaffold separately, and the drug release profiles were monitored by incubating a drug-loaded-scaffold in 1.0 ml PBS (pH=7.4) at 37° C. (n=3). At each time point, 1 ml of solution was collected and replaced with 1 ml of fresh PBS.

DOX Release Curve from the Premade Composite Scaffold in Two Months

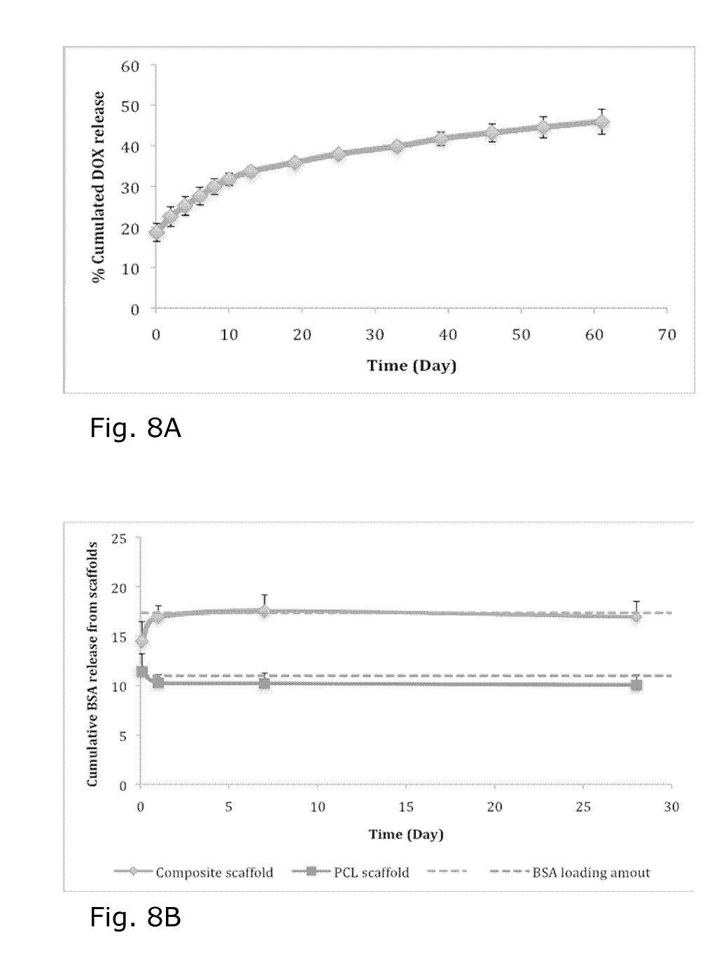

Cumulative drug release from the scaffolds was calculated and release curves were depicted in FIG. 8A.

The DOX loading efficiency on the composite scaffold was about 80.0%. DOX released rapidly from the scaffolds at the first 2.5 h and then slowly sustained released for 61 days. About 18.7% of the loading DOX was released from the scaffold at the first 3 hours. By day 61, about 46.0% DOX released from the scaffold.

BSA Release Curves from the PCL Scaffold and the Composite Scaffold in One Month

The composite scaffold has a higher loading efficiency for BSA compared to the PCL scaffold (FIG. 8B). The loading efficiency was 44.0% for the PCL scaffold and 69.4% for the composite scaffold.

BSA released rapidly at the first 2 hours from both the PCL scaffold and the composite scaffold. For the PCL scaffold, 104.1% of the loading BSA released from the scaffolds at the first 2 hours, that is, all of the loaded protein. For the composite scaffold, 83.4% of the loading BSA released at the first 2 hours and 101.1% released after 1 week.

Lysozyme Release Curves from PCL Scaffold and Composite Scaffold in One Month

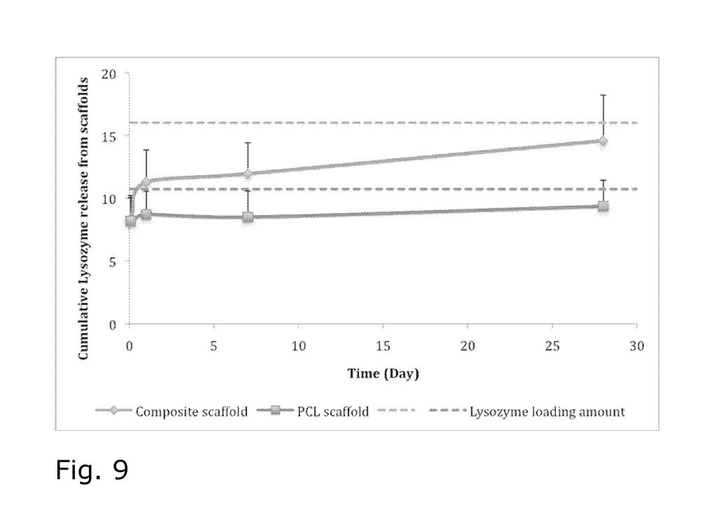

The composite scaffold has a higher loading efficiency for Lysozyme compared to the PCL scaffold (FIG. 9). The loading efficiency was 42.9% for the PCL scaffold and 64.0% for the composite scaffold.

PCL scaffold has a higher burst release rate compared to the composite scaffold. For the PCL scaffold, there was about 76.3% of the loading Lysozyme was released from the scaffold at the first 2 hours, and 87.1% released from the PCL scaffold after 4 weeks. For the composite scaffold, 51.7% of the loading Lysozyme released from the scaffold at the first 2 hours and 91.1% released after 4 weeks.

siRNA Delivery and Silencing Data

In this study, three different siRNA transfection reagents (oligofectamine, lipofectamine-2000 and chitosan nanoparticles) with siRNA targeted towards GFP or mismatch siRNA was loaded in the composite scaffolds. Three different cancer cell lines of Glioma cells (U251-eGFP), Human epithelial cervix carcinoma cell line (HeLa-eGFP) and human non-small cell lung carcinoma (H1299-eGFP) were grown beneath the scaffolds for 72 hours. Then the cells were collected and the GFP silencing was detected by flow cytometer.

HeLa cells stably expressing eGFP (HeLa-eGFP) were obtained by transfection with pEGFP-C1 (Clontech Laboratories, USA). A clone of the transfected pool was derived by G418 selection and cultivated in D-MEM (GIBCO-Invitrogen Corporation, USA) containing 10% FBS (foetal bovine serum) and 1% penicillin/streptomycin. The human lung cancer cell line H1299 produced to express EGFP stably (EGFP half-life 2 h) was a gift from Dr. Anne Chauchereau (CNRS, Villejuif, France). Cells were plated on multiwell 24-well plates (10⁵ cells/well) in RPMI media (containing 10% fetal bovine serum (FBS), 5% penicillin/streptomycin.

The lipofectamine and oligofectamine were purchased from Invitrogen. Chitosan particles were made by adding 20 μL 100 μM siRNA to 1 mL 0.72 mg/ml chitosan solution (250 kDa, 100% deacetylation, in 0.3M NaAc buffer, pH 5.5) whilst stirring and continuing the stirring for 1 hour before adding 200 uL 60% sucrose, then use it to coat scaffolds.

The transfection protocol for making siRNA loaded lipofectamine and oligofectamine particles is as follows:

Start by vortexing the transfection reagent and hand mixing the siRNA tube

In separate tubes:
Mix 15 μL 20 μM siRNA with 500 μL serum free medium by pipetting up & down
Mix 15 μL transfection reagent with 500 μL serum free medium by pipetting up & down
Incubate both tubes for 5 min at room temperature
Combine the content of the tubes and pipette up & down (or vortex briefly)
Wait 20 min at room temperature
Use (For example 50 μL in 200 μL serum containing medium in a 24 well giving an effective concentration of 50 nM siRNA)

### Notes

1. Both recipes can be scaled up or down according to the needed volume of particles just retain the same ratios of siRNA to transfection regent and medium.
2. If is lower concentration of siRNA is needed then add less to the cells for example add just 25 μL siRNA/transfection mixture to 225 μL medium containing serum to achieve an effective well concentration of 25 nM siRNA

The scaffolds were loaded with particles by pipetting the resulting suspension onto them followed by lyophilization at −20 degrees Celsius for 3 days.

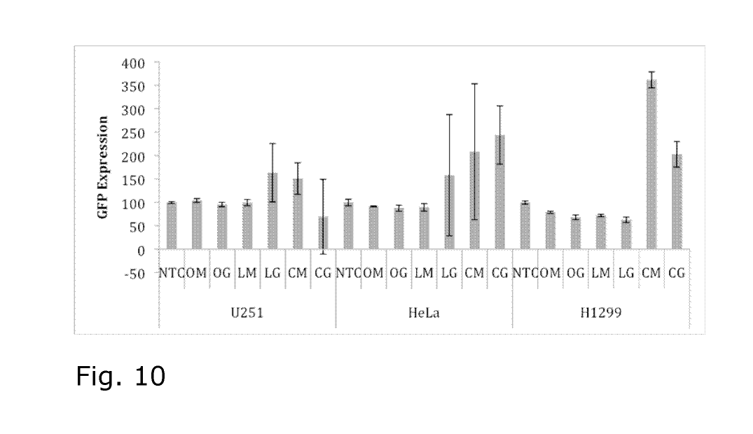

Results (FIG. 10) showed that the composite scaffold was able to be used for siRNA local delivery. Chitosan transfection reagent has higher silencing GFP effect for both Glioma cells (U251-eGFP) and human non-small cell lung carcinoma (H1299-eGFP) cells lines.

### REFERENCES

Di Martino, A., M. Sittinger, et al. (2005). “Chitosan: a versatile biopolymer for orthopaedic tissue-engineering.” Biomaterials 26(30): 5983-90.
El-Ghannam, A., K. Ricci, et al. (2010). “A ceramic-based anticancer drug delivery system to treat breast cancer.” Journal of materials science Materials in medicine.
Fröschle, G. W., J. Mählitz, et al. (1997). “Release of daunorubicin from polymethylmethacrylate for the improvement of the local growth control of bone metastasis animal experiments.” Anticancer Res 17(2A): 995-1002.
Habraken, W. J., J. G. Wolke, et al. (2007). “Ceramic composites as matrices and scaffolds for drug delivery in tissue engineering.” Adv Drug Deliv Rev 59(4-5): 234-48.

Huang, W., X. Shi, et al. (2010). “PHBV microspheres—PLGA matrix composite scaffold for bone tissue engineering.” Biomaterials 31(15): 4278-85.
Hutmacher, D. W., M. Sittinger, et al. (2004). “Scaffold-based tissue engineering: rationale for computer-aided design and solid free-form fabrication systems.” Trends Biotechnol 22(7): 354-62.
Itokazu, M., T. Sugiyama, et al. (1998). “Development of porous apatite ceramic for local delivery of chemotherapeutic agents.” Journal of biomedical materials research 39(4): 536-8.
Kumar, D., L. A. Kirshenbaum, et al. (2001). “Apoptosis in adriamycin cardiomyopathy and its modulation by probucol.” Antioxid Redox Signal 3(1): 135-45.
Lee, S. H. and H. Shin (2007). “Matrices and scaffolds for delivery of bioactive molecules in bone and cartilage tissue engineering.” Adv Drug Deliv Rev 59(4-5): 339-59.
Lee, Y. M., Y. J. Park, et al. (2000). “Tissue engineered bone formation using chitosan/tricalcium phosphate sponges.” J Periodontol 71(3): 410-7.
Lickorish, D., J. A. Ramshaw, et al. (2004). “Collagen-hydroxyapatite composite prepared by biomimetic process.” J. Biomed. Mater. Res. 68(1): 19-27.
Mei, N., G. Chen, et al. (2005). “Biocompatibility of Poly(epsilon-caprolactone) scaffold modified by chitosan—the fibroblasts proliferation in vitro.” Journal of Biomaterials Applications 19(4): 323-39.
Meijer, G. J., J. D. de Bruijn, et al. (2007). “Cell-based bone tissue engineering.” PLoS Med 4(2): e9.
Sohier, J., G. Daculsi, et al. “Porous beta tricalcium phosphate scaffolds used as a BMP-2 delivery system for bone tissue engineering.” J Biomed Mater Res A 92(3): 1105-14.
Taché, A., L. Gan, et al. (2004). “Effect of surface chemistry on the rate of osseointegration of sintered porous-surfaced Ti-6A1-4V implants.” Int J Oral Maxillofac Implants 19(1): 19-29.
Takemura, G. and H. Fujiwara (2007). “Doxorubicin-induced cardiomyopathy from the cardiotoxic mechanisms to management.” Progress in cardiovascular diseases.
Wenk, E., A. J. Meinel, et al. (2009). “Microporous silk fibroin scaffolds embedding PLGA microparticles for controlled growth factor delivery in tissue engineering.” Biomaterials 30(13): 2571-81.
Yuan, Q., J. Shah, et al. (2010). “Controlled and extended drug release behavior of chitosan-based nanoparticle carrier.” Acta biomaterialia 6(3): 1140-8.
Zhu, Y., C. Gao, et al. (2002). “Surface modification of polycaprolactone with poly(methacrylic acid) and gelatin covalent immobilization for promoting its cytocompatibility.” Biomaterials 23(24): 4889-95.

1. A three-dimensional hybrid scaffold capable of supporting cell growth and differentiation, and capable of controlled release of active pharmaceutical ingredients, wherein the scaffold comprises a first and a second biocompatible material, said first material shaped as a framework forming one or more open networks of voids, said second material comprising an ion exchange material, said ion exchange material being loaded with one or more active pharmaceutical ingredients;
wherein the ion exchange material is a clay mineral derivative comprising chitosan; and
wherein said second material is in the form of a porous foam.

2-20. (canceled)

21. The three-dimensional hybrid scaffold according to claim 1, wherein the scaffold is a 3D plotted scaffold.

22. The three-dimensional hybrid scaffold according to claim 21, wherein the clay mineral derivative is a Phyllosilicate derivative.

23. The three-dimensional hybrid scaffold according to claim 22, wherein the Phyllosilicate derivative is 2:1 Phyllosilicates or 1:1 Phyllosilicates.

24. The three-dimensional hybrid scaffold according to claim 1, wherein the ion exchange material is a Layered Double Hydroxide (LDH) derivative.

25. The three-dimensional hybrid scaffold according to claim 1, wherein the second biocompatible material comprises chitosan-tricalcium phosphate (TCP).

26. The three-dimensional hybrid scaffold according to claim 1, further comprising a binder.

27. The three-dimensional hybrid scaffold according to claim 1, wherein the second biocompatible material is in the form of a coating layer.

28. The three-dimensional hybrid scaffold according to claim 1, wherein the second material fills 0.001-100% of the total void volume of said open networks.

29. The three-dimensional hybrid scaffold according to claim 1, wherein the scaffold surface is coated with a natural or synthetic coating material.

30. The three-dimensional hybrid scaffold according to claim 29, wherein the scaffold surface is coated with a material selected from the group consisting of protein, peptides, nucleotides, and small interfering RNAs or mixtures thereof.

31. The three-dimensional hybrid scaffold according to claim 1, further comprising apatites.

32. The three-dimensional hybrid scaffold according to claim 1, wherein the scaffold is biodegradable.

33. A method of repairing or regenerating a tissue selected from the group consisting of bone, soft tissue, cartilage, brain and spinal cord tissue comprising proving the three-dimensional hybrid scaffold of claim 1 to a subject in need thereof.

34. A pharmaceutical composition comprising the three-dimensional hybrid scaffold according to claim 1.
